(19) United States
(12) Patent Application Publication
(10) Pub. No.: US 2006/0053686 A1
(43) Pub. Date: Mar. 16, 2006

(54) Pollen harvesting

(76) Inventor: Garry Wayne Halwas, Russell (CA)

Correspondence Address: ADE & COMPANY, 1700-360 MAIN STREET, WINNIPEG, MB, R3C3Z3, CA

(21) Appl. No.: 10/941,055

(22) Filed: Sep. 15, 2004

(51) Int. Cl. A01H 1/02

(52) U.S. Cl. 47/1.41

(57) ABSTRACT

Pollen is harvested from plants by mounting a suction nozzle on a vehicle such as a crop harvester or crop sprayer so as to be carried thereby and arranged on the vehicle so as to be located in the field adjacent the plants as the vehicle moves across the ground. A suction pump is mounted on a trailer behind the vehicle for communicating, though a series of ducts passing along side the vehicle, an air stream through the nozzle such that the air stream causes the pollen from the plants to be drawn into the nozzle. A separating system including cyclones and filtration bags is mounted on the trailer for extracting the pollen from the air stream.

### BACKGROUND OF THE INVENTION

Pollen is known to have nutritional and potential pharmaceutical properties and is used in some products for these purposes. Pollen is of course very light and produced in relatively low quantities so that its harvesting is problematic. Bees of course collect pollen but only on a very low level so that their activities cannot be in anyway considered for commercial harvesting of pollen.

Conventionally pollen is harvested in basically the same way as other crop materials by harvesting the crop, collecting the crop after cutting the crop in the field and by separating from the collected crop the pollen content. This is conventionally a highly expensive and low productively process since much of the pollen is lost during the handling and since the amount produced for a given quantity of the crop material is very low. However up till now there has been no suitable technique for harvesting pollen other than this unsatisfactory technique leading to a high cost of the pollen. There is therefore an opportunity for alternative harvesting techniques to be developed which can lead to significant increase in productivity and a high profit margin.

### SUMMARY OF THE INVENTION

It is one object of the invention to provide a method and apparatus for harvesting pollen.

According to one aspect of the invention there is provided a method for harvesting pollen from plants comprising:

providing a vehicle arranged to travel over a field containing growing plants carrying the pollen;

providing on the vehicle a suction nozzle so as to be carried thereby and arranged on the vehicle so as to be located in the field adjacent the plants as the vehicle moves across the ground;

providing a suction pump for generating an air stream through the nozzle such that the air stream causes the pollen from the plants to be drawn into the nozzle;

and causing the air stream to pass through a separating system for extracting the pollen from the air stream.

In one preferred arrangement, the vehicle is a harvester for cutting down the plants for harvesting and the pollen is extracted during the cutting. In this case the suction nozzle is preferably located above a cutter bar of the crop harvester. The cutter bar may be of the sickle knife or rotary type or other type well known to persons skilled in this art

In an alternative, the vehicle is a crop treatment machine such as a sprayer for passing over the growing crops.

Preferably where the machine is designed to act in a swath across the field, the suction nozzle is elongate across a full operating width of the vehicle.

Preferably there is provided a series of ducts at spaced position across the width of the vehicle and each extending from the nozzle to the suction pump.

Preferably the separation system is carried on a trailer arranged to be towed behind the vehicle.

Preferably the suction pump is also carried on the trailer.

Preferably the separation system comprises at least one cyclone and generally more than one cyclone arranged side by side on the trailer.

Preferably each cyclone has a collection container at the bottom and a permeable filtration bag at the top for escape of air and entrapment of particles.

According to a second aspect of the invention there is provided an apparatus for harvesting pollen from plants comprising:

a vehicle arranged to travel over a field containing growing plants carrying the pollen;

a suction nozzle mounted on the vehicle so as to be carried thereby and arranged on the vehicle so as to be located in the field adjacent the plants as the vehicle moves across the ground;

a suction pump for generating an air stream through the nozzle such that the air stream causes the pollen from the plants to be drawn into the nozzle;

and a separating system for extracting the pollen from the air stream.

According to a second aspect of the invention there is provided an apparatus for harvesting pollen from plants comprising:

a suction nozzle arranged to be mounted on a vehicle arranged to travel over a field containing growing plants carrying the pollen so as to be carried thereby and arranged on the vehicle so as to be located in the field adjacent the plants as the vehicle moves across the ground;

a trailer arranged to be towed behind the vehicle;

a suction pump mounted on the trailer and connected to the suction duct through one of more suction ducts for generating an air stream through the nozzle such that the air stream causes the pollen from the plants to be drawn into the nozzle;

and a separating system mounted on the trailer for extracting the pollen from the air stream.

### BRIEF DESCRIPTION OF THE DRAWINGS

One embodiment of the invention will now be described in conjunction with the accompanying drawings in which:

FIG. 1 is a top plan view of an apparatus according to the present invention for carrying out a method of harvesting pollen.

FIG. 2 is a side elevational view of the apparatus of FIG. 1.

In the drawings like characters of reference indicate corresponding parts in the different figures.

### DETAILED DESCRIPTION

The apparatus comprises a suction nozzle 10 for mounting on a header 11 of a harvesting machine generally indicated at 12. The harvesting machine comprises a cab 13 carried on a tractor unit 14 with front wheels 15 and rear wheels 16. The tractor unit maybe of different types but typically is a tractor unit of the type known as a self-propelled swather in which the tractor unit is driven by the front wheels with the rear wheels being castor wheels. At the forward end of the tractor unit is provided a suitable support mechanism schematically indicated at 16 for supporting the header 11. The link 17 is shown only schematically since many different arrangements are provided depending upon the different manufacturer and depending upon the type of header to be attached.

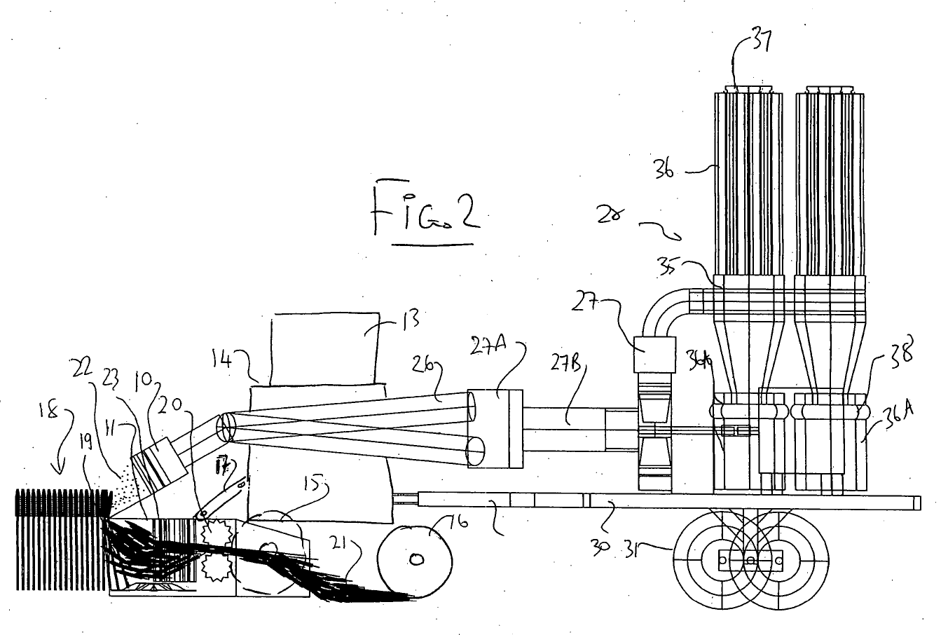

The header has a width significantly greater than that of the tractor unit to provide a cutting action in swath across the field carrying a crop 18 to be harvested. In the embodiment shown in FIG. 2, the header 11 is of the type including a series of rotary cutters 19 at spaced positions across the width of the header. Such rotary cutters are well known and manufactured by a number of different manufacturers. The rotary cutters are arranged so that they tend to carry the crop inwardly to a center discharge area where the crop is collated and passed through a pair of conditioning rollers 20 to form a swath 21 which is laid in the field between the wheels of the tractor and behind the header,

In the embodiment shown the suction nozzle 10 is mounted on the top of the header 11 by suitable mounting arrangements so that an open mouth 22 of the suction nozzle is located above a top wall 23 of the header 11. The top wall of the header provides a cover under which the crop material tends to pass after the cutting action of the rotary cutters. It is common that the height of the crop prior to cutting is above the top wall 23 although this is not necessarily so depending upon the type of crop concerned. The suction nozzle 10 and its mouth 22 forms a slot across the width of the header. Typically the slot may have a height of the order of six to twelve inches. The mounting of the slot is preferably as far forward as possible but it maybe desirable to locate the mounting structure for the nozzle at the rear of the of the header so as to provide a better weight balance for the system and to prevent the weight of the nozzle being counter levered too far forwardly from the supporting links 17.

The nozzle thus provides a mouth across the full width of header so any pollen being released from the crop as it is shaken and activated by the cutting action tends to rise from the crop and pass over the top cover 23 into the nozzle 10.

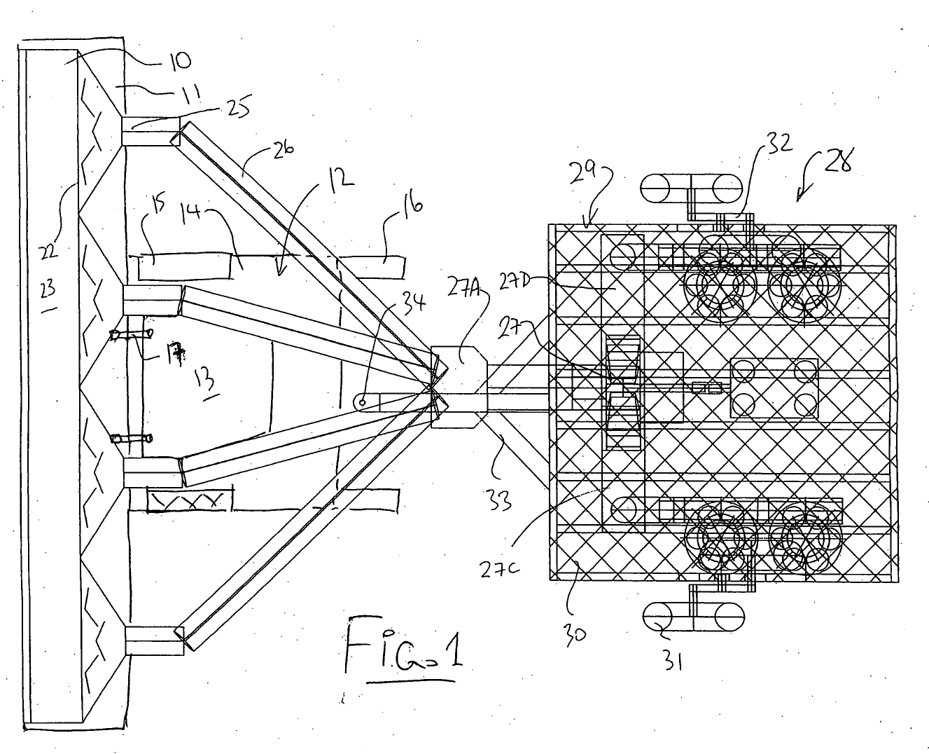

As best shown in FIG. 1, the nozzle is formed in four separate sections which converge inwardly and rearwardly from the open mouth 22 to a discharge duct 25. The number of sections can of course vary depending upon the width of the header. In the embodiment concerned where the header typically has a with of the order of sixteen to twenty feet, a division of the nozzle into four separate sections is convenient to provide four discharge ducts 25 extending rearwardly from the nozzle.

Each duct 25 is connected to a respective transfer duct 26 which extends from the rear end of the discharge duct to a suction pump 27 behind the vehicle 12. Thus each of the ducts 26 extends along a side of the tractor and around the rear of the tractor to a single suction pump located behind the tractor. Each duct 26 therefore passes along side or around the out side of the cab suitably draped over the vehicle itself to be supported thereby. Flexible hoses of a type well known an commercially available can be used for providing the transfer duct.

The suction pump 27 together with a separating system generally indicated at 28 are both mounted on a trailer 29 trailed behind the vehicle 12. The trailer 29 comprises a flat bed 30 carried on suitable ground wheels 31 on a suspension system 32. The trailer includes a hitch section 33 which extends forwardly from the flat bed 30 to a front hitch 34 which is attached to a hitch coupling at the rear of the vehicle. Thus the trailer carrying the suction pump 27 and the separation system 28 is trailed behind the vehicle and can be readily transported behind the vehicle with the relatively light weight of the suction system and the ducts carried on the vehicle itself and on the header. The suction pump 27 is mounted on the forward part of the bed 30 and behind that is mounted the separation section 28. The separation section comprises four cyclones 35 at the bottom of which is mounted for each cyclone a respective collection container 36 so that materials heavier than the air tend to collect in the cyclone in well known manner and discharge downwardly for collection into the containers. Air discharged upwardly enters a respective filtration bag 36 which stands upwardly form the top of the cyclone and is supported on a top support plat 37 standing upwardly from the cyclone and carried on a suitable support structure (not shown). Thus air pumped from the pump 27 carrying the collected pollen and other materials is collected within the container and filtration system so that the pollen tend to collect in the container after falling downwardly either from the filtration system or form the separation action in the cyclone. The pollen is thus collected in the container which can be removed from the bottom of the cyclone by a fastening system 38 which connects the container 36A to the bottom of the cyclone 35.

A the rear of the ducts 26 is provided an manifold 27A which provides a coupling pipe 27B extending rearwardly to the inlet side of the pump 27. The outlet side of the pump 27 provides two outlet ducts 27C and 27D each of which communicates to a respective pair of the four cyclones which are arranged in series along the respective duct. Thus the air flow generated by the pump carries the material expelled from the crop and rising above the crop and over the top of the header into the suction nozzle from where it is carried through the pump into the separation system.

Since various modifications can be made in my invention as herein above described, and many apparently widely different embodiments of same made within the spirit and scope of the Claims without department from such spirit and scope, it is intended that all matter contained in the accompanying specification shall be interpreted as illustrative only and not in a limiting sense.

1. A method for harvesting pollen from plants comprising:
providing a vehicle arranged to travel over a field containing growing plants carrying the pollen;
providing on the vehicle a suction nozzle so as to be carried thereby and arranged on the vehicle so as to be located in the field adjacent the plants as the vehicle moves across the ground;
providing a suction pump for generating an air stream through the nozzle such that the air stream causes the pollen from the plants to be drawn into the nozzle;
and causing the air stream to pass through a separating system for extracting the pollen from the air stream.

2. The method according to claim 1 wherein the vehicle is harvester for cutting down the plants for harvesting and the pollen is extracted during the cutting.

3. The method according to claim 1 wherein the vehicle is a crop treatment machine for passing over the growing crops.

4. The method according to claim 3 wherein the crop treatment machine is a sprayer.

5. The method according to claim 2 wherein the suction nozzle is located above a cutter bar of the crop harvester.

6. The method according to claim 1 wherein the suction nozzle is elongate across a full operating width of the vehicle.

7. The method according to claim 1 wherein there is provided a series of ducts at spaced position across the width of the vehicle and each extending from the nozzle to the suction pump.

8. The method according to claim 1 wherein the separation system is carried on a trailer arranged to be towed behind the vehicle.

9. The method according to claim 8 wherein the suction pump is carried on the trailer.

10. The method according to claim 1 wherein the separation system comprises at least one cyclone.

11. The method according to claim 10 wherein the separation system comprises a plurality of cyclones arranged side by side on the trailer.

12. The method according to claim 11 wherein each cyclone has a collection container at the bottom and a permeable filtration bag at the top for escape of air and entrapment of particles.

13. Apparatus for harvesting pollen from plants comprising:
a vehicle arranged to travel over a field containing growing plants carrying the pollen;
a suction nozzle mounted on the vehicle so as to be carried thereby and arranged on the vehicle so as to be located in the field adjacent the plants as the vehicle moves across the ground;
a suction pump for generating an air stream through the nozzle such that the air stream causes the pollen from the plants to be drawn into the nozzle;
and a separating system for extracting the pollen from the air stream.

14. Apparatus for harvesting pollen from plants comprising:
a suction nozzle arranged to be mounted on a vehicle arranged to travel over a field containing growing plants carrying the pollen so as to be carried thereby and arranged on the vehicle so as to be located in the field adjacent the plants as the vehicle moves across the ground;
a trailer arranged to be towed behind the vehicle;
a suction pump mounted on the trailer and connected to the suction duct through one of more suction ducts for generating an air stream through the nozzle such that the air stream causes the pollen from the plants to be drawn into the nozzle;
and a separating system mounted on the trailer for extracting the pollen from the air stream.
